(19) United States
(12) Patent Application Publication
(10) Pub. No.: US 2016/0291018 A1
(43) Pub. Date: Oct. 6, 2016

(54) USE OF PCSK9 AND LDL-R ACTIVITY FOR TREATING CARDIOVASCULAR RISK

(71) Applicant: True Health Diagnostics LLC, Frisco, TX (US)

(72) Inventor: Rebecca E. Caffrey, SR., N. Chesterfield, VA (US)

(21) Appl. No.: 15/091,818

(22) Filed: Apr. 6, 2016

Related U.S. Application Data
(60) Provisional application No. 62/143,452, filed on Apr. 6, 2015.

(51) Int. Cl. G01N 33/573; G01N 33/92

(52) U.S. Cl. CPC G01N 33/573; G01N 33/92; G01N 2333/96433; G01N 2800/32; G01N 2800/50

(57) ABSTRACT

The present disclosure provides methods of assessing cardiovascular risk in a subject and/or of treating a subject having or at risk of developing a cardiovascular disease or disorder. In some embodiments, the method comprises determining an activity level of PCSK9 and/or a level of PCSK9 in a sample obtained from the subject, and initiating or modifying a treatment regimen.

### PRIORITY CLAIM

This application claims priority to U.S. Provisional Patent Application Ser. No. 62/143,452, filed Apr. 6, 2015, which is incorporated herein by reference in its entirety and relied upon.

### FIELD

The present disclosure provides methods of assessing cardiovascular risk in a subject, and/or of treating a subject having or at risk of developing a cardiovascular disease or disorder.

### BACKGROUND

LDL-p and LDL-c levels are thought to be related to a subject's cardiovascular risk. However, current efforts to predict risk do not consider contributions from LDL receptor (“LDL-r”) and Proprotein Convertase Subtilisin/Kexin type 9 (“PCSK9”) on LDL-p and LDL-c levels. Notably, biological activity of PCSK9 and LDL-r vary from subject to subject, partially due to genetic factors. Existing assays for PCSK9 and LDL-r typically do not account for these genetic factors, resulting in inefficient and/or inaccurate results. Such inaccuracies may lead to selection of a sub-optimal therapeutic regimen or, worse, no therapeutic regimen when one should be initiated. The need for more accurate assessment of cardiovascular risk is apparent.

### SUMMARY

In some embodiments, the present disclosure provides a method of treating a subject at risk of developing a cardiovascular disease or disorder, the method comprising contacting PCSK9 with LDL-r under conditions effective to form a PCSK9:LDL-r complex, wherein at least one of the PCSK9 or LDL-r is derived from the subject; determining a binding affinity of the PCSK9 and the LDL-r based on said contacting; comparing the determined binding affinity of the PCSK9 and the LDL-r to a reference value; and determining a percent PCSK9 function in the subject based on the step of comparing, wherein the percent PCSK9 function is the percent binding affinity of the PCSK9 to the LDL-r relative to the reference value; determining a level of PCSK9 in the subject; and treating the subject for the cardiovascular disease or disorder if the subject is at an elevated risk for developing cardiovascular disease based on the determined percent PCSK9 function and the determined PCSK9 level.

In some embodiments, the present disclosure provides a solid support comprising isolated PCSK9 and isolated low density lipoprotein receptor (“LDL r”), wherein at least one of the isolated PCSK9 or the isolated LDL-r is derived from a biological sample and at least one of the isolated PCSK9 or the isolated LDL-r is coupled to the support.

In some embodiments, the present disclosure provides a kit comprising a solid support as disclosed herein (e.g., a solid support comprising isolated PCSK9 and isolated LDL-r, wherein at least one of the isolated PCSK9 or the isolated LDL-r is derived from a biological sample and at least one of the isolated PCSK9 or the isolated LDL-r is coupled to the support) and a reagent for detecting the PCSK9 and/or the LDL-r.

These and other embodiments are described in greater detail below.

### DETAILED DESCRIPTION

There are two physical structures for measurement of absolute quantity and biological activity.

An ELISA or similar technology (nephelometry, electrophoresis) that utilizes a binding capture agent for PCSK9 and LDL-R derived from patient samples that allows for capture and absolute quantitation of amounts of these proteins that are present in the biological samples. Example: A sandwich ELISA wherein antibodies against the respective ligands are bound to a solid support, and a reporter antibody to another epitope on the ligands which is conjugated to a reporter molecule that allows for measurement of quantity via fluorescence or color change, and a detector to measure the amount of protein present relative to controls of known concentration.

A modified ELISA or similar technology (nephelometry) that utilizes a binding capture agent for PCSK9 and LDL-R derived from patient samples that allows for capture and measurement of affinity of the ligands for one another, thus deriving a measurement for functional biological interaction of these ligands in a given patient. Example: Native PCSK9 from patient serum is captured by an antibody on a solid support, and native LDL-R isolated from patient blood cell membranes is allowed to interact with the bound PCSK9. The affinity of the interaction determines the amount that will bind and can be detected by a secondary antibody conjugated to a reporter molecule as above that can be measured by a detector.

Comparison of the quantities of each of the 2 proteins to a reference range in order to classify them as low, normal or high in expression, which shall correlate with known cardiovascular risk. The measurements will be further compared to one another, and each of these values may be further compared to LDL-P and LDL-C measurements from the patient sample. Comparison may be absolute values or ratios derived from absolute values. These measurements will also be compared to reference ranges in order to classify a given patient's cardiovascular risk levels.

Clinical action will be taken based on this information to affect optimal treatment of LDL and reduction in cardiovascular risk profile based on the information provided by this combination of assays. This clinical guidance may take the form of a nomogram or software tool.

Clinical actions taken based on this information may include prescribing one or more types of drugs that specifically inhibit the interaction of these 2 molecules in vivo in order to lower LDL cholesterol and cardiovascular risk. This action (or lack thereof if values are normal) may be combined with statin therapy. Actions may also include recommendations for the patient to change diet, lifestyle, exercise patterns, etc. These clinical actions may be undertaken by any healthcare provider in a counseling setting, via written communication, or via electronic communication.

In some embodiments, the present disclosure provides a method of treating a subject at risk of developing a cardiovascular disease or disorder, the method comprising contacting PCSK9 with LDL-r under conditions effective to form a PCSK9:LDL-r complex, wherein at least one of the PCSK9 or LDL-r is derived from the subject; determining a binding affinity of the PCSK9 and the LDL-r based on said contacting; comparing the determined binding affinity of the PCSK9 and the LDL-r to a reference value; and determining a percent PCSK9 function in the subject based on the step of comparing, wherein the percent PCSK9 function is the percent binding affinity of the PCSK9 to the LDL-r relative to the reference value; determining a level of PCSK9 in the subject; and treating the subject for the cardiovascular disease or disorder if the subject is at an elevated risk for developing cardiovascular disease based on the determined percent PCSK9 function and the determined PCSK9 level. In some embodiments, the method further comprises deriving a cardiovascular risk index value for the subject based on the determined percent PCSK9 function of the subject and the measured absolute concentration of PCSK9 in the subject; optionally comparing the derived cardiovascular risk index value with a reference cardiovascular risk index value range; and determining if the subject is at an elevated risk for developing cardiovascular disease based on the derived cardiovascular risk index value or the comparison of the derived cardiovascular risk index value to the reference cardiovascular risk index value. In some embodiments, the PCSK9 has been isolated from a biological sample from the subject. In some embodiments, the biological sample is selected from the group consisting of human biological matrices, urine, plasma, and serum. In some embodiments, the LDL-r has been isolated from a biological sample from the subject. In some embodiments, the biological sample is selected from the group consisting of human biological matrices, urine, plasma, and serum. In some embodiments, the LDL-r or PCSK9 is coupled to a solid support. In some embodiments, the LDL-r and/or the PCSK9 is coupled to a detectable moiety. In some embodiments, the step of measuring comprises detecting the detectable moiety. In some embodiments, the reference value is the binding affinity of fully-functioning PCSK9 to fully-functioning LDL-r. In some embodiments, the subject is a mammal. In some embodiments, the step of deriving a cardiovascular risk index value for the subject comprises calculating the product of the determined percent PCSK9 function of the subject and the measured absolute concentration of PCSK9 in the subject. In some embodiments, the step of determining the binding affinity comprises SPR, ELISA, NMR, mass spectrometry, chromatography, or spectroscopy. In some embodiments, the step of determining a level of PCSK9 is carried out using SPR, ELISA, NMR, mass spectrometry, chromatography, or spectroscopy. In some embodiments, a therapy regimen is selected based at least on the elevated risk for developing cardiovascular disease. In some embodiments, the selected therapy regimen comprises a drug and/or a supplement. In some embodiments, the selected therapy regimen comprises a PCSK9 inhibitor. In some embodiments, the selected therapy regimen comprises a drug selected from the group consisting of: an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof In some embodiments, the selected therapy regimen comprises giving recommendations on making or maintaining lifestyle choices based on the results of said determining. In some embodiments, the lifestyle choices involve changes in diet, changes in exercise, reducing or eliminating smoking, or a combination thereof. In some embodiments, the step of determining if the subject is at an elevated risk for developing cardiovascular disease further comprises assigning the subject to a risk category selected from the group consisting of: high risk for developing or having cardiovascular disease, intermediate risk for developing or having cardiovascular disease, and low risk for developing or having cardiovascular disease. In some embodiments, the method further comprises determining a level of LDL-P and/or LDL-C in the biological sample(s).

In some embodiments, the present disclosure provides a solid support comprising isolated PCSK9 and isolated low density lipoprotein receptor (“LDL r”), wherein at least one of the isolated PCSK9 or the isolated LDL-r is derived from a biological sample and at least one of the isolated PCSK9 or the isolated LDL-r is coupled to the support.

In some embodiments, the present disclosure provides a kit comprising a solid support as disclosed herein (e.g., a solid support comprising isolated PCSK9 and isolated LDL-r, wherein at least one of the isolated PCSK9 or the isolated LDL-r is derived from a biological sample and at least one of the isolated PCSK9 or the isolated LDL-r is coupled to the support) and a reagent for detecting the PCSK9 and/or the LDL-r. In some embodiments, the reagent is a labeled reagent. In some embodiments, the PCSK9 is coupled to the solid support by an antibody selective for PCSK9 or a derivative thereof, a fragment of an antibody selective for PCSK9 or derivative thereof, an aptamer selective for PCSK9 or a derivative thereof, or a ligand selective for PCSK9 or derivative thereof. In some embodiments, the LDL-r is coupled to the support by an antibody selective for LDL-r or a derivative thereof, a fragment of an antibody selective for LDL-r or derivative thereof, an aptamer selective for LDL-r or a derivative thereof, or a ligand selective for LDL-r or derivative thereof. In some embodiments, the PCSK9 is isolated from a biological sample from a subject. In some embodiments, the PCSK9 is recombinant PCSK9. In some embodiments, the LDL-r is isolated from a biological sample from a subject. In some embodiments, the LDL-r is recombinant LDL-r. In some embodiments, the solid support comprises a gel, a 96-well plate, a non-96-well-configured plate, a non-denaturing electrophoretic gel, a bead, a slide, a capillary, a microfluidic device, a support used for chromatography, a MALDI surface, a SELDI surface, or a lipid bilayer interferometry device.

In some embodiments, a solid support comprises LDL-R from a subject as a primary ligand. In such embodiments, the solid support may be contacted by a sample (e.g., a biological sample from the subject) comprising PCSK9 for a period of time sufficient to form a PCSK9:LDL-R complex. In some embodiments, the solid support-PCSK9:LDL-R complex may be contacted with a secondary antibody specific for PCSK9, optionally including a reporter molecule (e.g., a label).

In some embodiments, the method comprises determining an absolute level of a protein (e.g., PCSK9 and/or LDL-r) in a biological sample. In other embodiments, a determined level of a protein in a biological sample is relative to a standard.

Determination of protein levels in a biological sample may be performed by any technique known to those skilled in the art, including but not limited to ELISA, immunoprecipitation, Western blot, mass spectrometry, other electrophoresis, nephelometry, RIA, and the like.

Determination of protein interaction affinity may be performed by any technique known to those skilled in the art, including but not limited to modified ELISA on plates or beads or other solid support, electrophoresis, nephelometry, and the like.

In some embodiments, the solid supports and/or methods described herein feature non-antibody capture ligands having specific affinity for PCSK9 and/or LDL-R. In some embodiments, a non-antibody capture ligand having specific affinity for PCSK9 is used in place of the antibody selective for PCSK9. In some embodiments, a non-antibody capture ligand having specific affinity for LDL-r is used in place of the antibody selective for LDL-r.

A drug may be introduced to the affinity assay in order to assess potential efficacy through measurement of changes in affinity compared to the measured affinities in samples where no drug is present. This companion diagnostic application may be used, for example, to guide choice of the most appropriate drug for that subject given the subject's specific phenotype.

Actual LDL-P contacted from a subject's biological sample may be added to functional affinity assays to gain further information on the biological function in vivo in a given patient.

In some embodiments, a method of assessing treatment efficacy and optionally guiding decision-making comprises determining a percent PCSK9 function and a level of PCSK9 in biological samples obtained over time. In some embodiments, the method further comprises determining a level of a drug or supplement in the biological sample(s) for assessing dosing and the subject's compliance with the drug regimen. Determining percent LSCK9 function and the level of PCSK9 in the biological samples may be accomplished using methods as disclosed herein. In some embodiments, the method further comprises recommending or affecting a change in the subject's treatment based at least in part on the determined percent PCSK9 function and level of PCSK9 in the biological sample(s), for example based at least in part on changes in the percent LCSK9 function and/or changes in the level of PCSK9 in the biological samples over time.

We claim:

1. A method of treating a subject at risk of developing a cardiovascular disease or disorder, the method comprising:
contacting PCSK9 with LDL-r under conditions effective to form a PCSK9:LDL-r complex, wherein at least one of the PCSK9 or LDL-r is derived from a subject;
determining a binding affinity of the PCSK9 and the LDL-r based on said contacting;
comparing the determined binding affinity of the PCSK9 and the LDL-r to a reference value;
determining a percent PCSK9 function in the subject based on the step of comparing, wherein the percent PCSK9 function is the percent binding affinity of the PCSK9 to the LDL-r relative to the reference value;
determining a level of PCSK9 in the subject; and
treating the subject for the cardiovascular disease or disorder if the subject is at an elevated risk for developing cardiovascular disease based on the determined percent PCSK9 function and the determined PCSK9 level.

2. The method according to claim 1 further comprising:
deriving a cardiovascular risk index value for the subject based on the determined percent PCSK9 function of the subject and the measured absolute concentration of PCSK9 in the subject;
optionally comparing the derived cardiovascular risk index value with a reference cardiovascular risk index value range; and
determining if the subject is at an elevated risk for developing cardiovascular disease based on the derived cardiovascular risk index value or the comparison of the derived cardiovascular risk index value to the reference cardiovascular risk index value.

3. The method according to claim 1, wherein the PCSK9 has been isolated from a biological sample from the subject.

4. The method according to claim 3, wherein the biological sample is selected from the group consisting of human biological matrices, urine, plasma, and serum.

5. The method according to claim 1, wherein the LDL-r has been isolated from a biological sample from the subject.

6. The method according to claim 5, wherein the biological sample is selected from the group consisting of human biological matrices, urine, plasma, and serum.

7. The method according to claim 1, wherein the LDL-r or PCSK9 is coupled to a solid support.

8. The method according to claim 1, wherein the LDL-r and/or the PCSK9 is coupled to a detectable moiety.

9. The method according to claim 8, wherein the step of measuring comprises detecting the detectable moiety.

10. The method according to claim 1, wherein the reference value is the binding affinity of fully-functioning PCSK9 to fully-functioning LDL-r.

11. The method according to claim 1, wherein the subject is a mammal.

12. The method according to claim 2, wherein the step of deriving a cardiovascular risk index value for the subject comprises calculating the product of the determined percent PCSK9 function of the subject and the measured absolute concentration of PCSK9 in the subject.

13. The method according to claim 1, wherein the step of determining the binding affinity comprises SPR, ELISA, NMR, mass spectrometry, chromatography, or spectroscopy.

14. The method according to claim 1, wherein the step of determining a level of PCSK9 is carried out using SPR, ELISA, NMR, mass spectrometry, chromatography, or spectroscopy.

15. The method according to claim 2, wherein a therapy regimen is selected based at least on the elevated risk for developing cardiovascular disease.

16. The method according to claim 15, wherein the selected therapy regimen comprises a drug and/or a supplement.

17. The method according to claim 15, wherein the selected therapy regimen comprises a PCSK9 inhibitor.

18. The method according to claim 15, wherein the selected therapy regimen comprises a drug selected from the group consisting of: an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof.

19. The method according to claim 15, wherein the selected therapy regimen comprises giving recommendations on making or maintaining lifestyle choices based on the results of said determining.

20. The method according to claim 19, wherein the lifestyle choices involve changes in diet, changes in exercise, reducing or eliminating smoking, or a combination thereof.

21. The method according to claim 2, wherein the step of determining if the subject is at an elevated risk for developing cardiovascular disease further comprises assigning the subject to a risk category selected from the group consisting of: high risk for developing or having cardiovascular disease, intermediate risk for developing or having cardiovascular disease, and low risk for developing or having cardiovascular disease.

22. The method of claim 1 further comprising determining a level of LDL-P and/or LDL-C in the biological sample(s).

23. A solid support comprising isolated PCSK9 and isolated low density lipoprotein receptor (“LDL-r”), wherein at least one of the isolated PCSK9 or the isolated LDL-r is derived from a biological sample and at least one of the isolated PCSK9 or the isolated LDL-r is coupled to the support.

24. A kit comprising the solid support of claim 23 and a reagent for detecting the PCSK9 and/or the LDL-r.

25. The kit according to claim 24, wherein the reagent is a labeled reagent.

26. The kit according to claim 24, wherein the PCSK9 is coupled to the solid support by an antibody selective for PCSK9 or a derivative thereof, a fragment of an antibody selective for PCSK9 or derivative thereof, an aptamer selective for PCSK9 or a derivative thereof, or a ligand selective for PCSK9 or derivative thereof.

27. The kit according to claim 24, wherein the LDL-r is coupled to the support by an antibody selective for LDL-r or a derivative thereof, a fragment of an antibody selective for LDL-r or derivative thereof, an aptamer selective for LDL-r or a derivative thereof, or a ligand selective for LDL-r or derivative thereof.

28. The kit according to claim 24, wherein the PCSK9 is isolated from a biological sample from a subject.

29. The kit according to claim 24, wherein the PCSK9 is recombinant PCSK9.

30. The kit according to claim 24, wherein the LDL-r is isolated from a biological sample from a subject.

31. The kit according to claim 24, wherein the LDL-r is recombinant LDL-r.

32. The kit according to claim 24, wherein the solid support comprises a gel, a 96-well plate, a non-96-well-configured plate, a non-denaturing electrophoretic gel, a bead, a slide, a capillary, a microfluidic device, a support used for chromatography, a MALDI surface, a SELDI surface, or a lipid bilayer interferometry device.
